(19) United States
(12) Patent Application Publication
(10) Pub. No.: US 2007/0099854 A1
(43) Pub. Date: May 3, 2007

(54) Use of pentadienoic acid derivatives for the treatment of hyperuricemia

(76) Inventors: Robert Boizel, Grenoble (FR); Pascale Fouqueray, Paris (FR); Daniel Guerrier, Saint Genis Laval (FR); Jean-Jacques Zeiller, Lyon (FR); Bertrand Brutzkus, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/580,602

(22) PCT Filed: Nov. 2, 2004
(86) PCT No.: PCT/EP04/12381

§ 371 Date: May 25, 2006

Related U.S. Application Data
(60) Provisional application No. 60/527,773, filed on Dec. 9, 2003.

(30) Foreign Application Priority Data
Nov. 28, 2003 (EP) 03292973.9

(51) Int. Cl. A61K 31/7048; A61K 31/382; A61K 31/353

(52) U.S. Cl. 514/27; 514/431; 514/432; 514/450; 514/456

(57) ABSTRACT

The use of a pentadienoic acid derivative of formula (I) for the preparation of a medicament for the prevention or treatment of hyperuricemia and/or one or several associated disorders or diseases, and/or for reducing the serum uric acid level of a subject. Medical compositions for these prevention and/or treatment, comprising such a pentadienoic acid derivative.

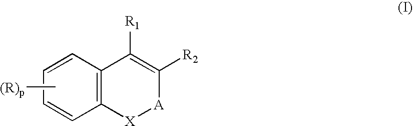

### FIELD OF THE INVENTION

The present invention relates to methods for decreasing plasma uric acid levels and treating gout related conditions, using pentadienoic acid derivatives, which have been identified as potent oral hypouricemic agents.

It also relates to the use of these derivatives for preparing medicaments for these methods, and to new medicaments for these purposes.

### BACKGROUND OF THE INVENTION

Uric acid is an end product of purine nucleotide catabolism in humans. Most mammals, but not humans, express the enzyme uricase, which further degrades uric acid to allantoin. Consequently, statistically normal uric acid levels in men and premenopausal women (7 mg per decilitre or 420 μmol/litre and 6 mg per decilitre or 360 μmol/litre respectively) are close to the limits of urate solubility (approximately 7 mg/decilitre at 370) in vitro, imposing a delicate physiologic urate balance. Uric acid is a weak organic acid. In serum condition of pH 7.40 and temperature 37° C., about 98% of uric acid is ionised as monosodium urate.

Hyperuricemia in humans is common and becomes more common with increased age, diverse pathological states and the use of some medications. Increased serum urate levels may be due to enhanced uric acid production and/or reduced renal excretion of uric acid. Uric acid overproduction may be related to excessive dietary purine intake, specific disease states (malignancy, psoriasis), increased turn over of ATP or inherited enzyme defects. Renal underexcretion of uric acid may be related to defects in the renal handling of uric acid, reduced glomerular filtration of urate or altered reabsorption—secretion by the proximal tubule.

Hyperuricemia is a metabolic disturbance that may lead to gout, which is a common medical problem, affecting at least 1 percent of men in Western countries. Increased levels of urate may leads to precipitation of urate crystals and tissue deposition of urate, leading to other manifestations of gout: attacks of acute inflammatory arthritis, tophaceous deposition of urate crystals in and around joints, chronic arthritis, deposition of urate crystals in renal parenchyma, and urolithiasis (all, either alone or in combination). The incidence of gouty arthritis is increased 5 fold in subjects with a serum urate level of 7 to 8.9 mg per decilitre and up to 50 fold in subjects with a serum urate level of at least 9 mg per decilitre (530 μmol per liter). Patients with gout may develop renal insufficiency and end stage renal disease. The renal disease, which has been termed “gouty nephropathy”, is characterized by a chronic interstitial nephropathy, which is promoted by medullary deposition of monosodium urate. In the vast majority of patients with gout (80-90%), increased serum urate levels are related to a diminished renal excretion of uric acid.

On the other hand, secondary hyperuricemia, drug related (i.e. diuretics, immunosuppressive and cytotoxic agents), or related to diverse medical conditions (i.e. various nephropathies, myeloproliferative disorders, conditions associated with insulin resistance and in transplant recipients) may also worsen kidney function leading to chronic and acute renal failure. Overproduction of urate and acid urine also increase the risk of calcium oxalate urolithiasis.

All clinical data and the management of hyperuricemia and gout are supported by references in Oxford Textbook of Clinical Nephrology, The Kidney (Brenner & Rector's), Renal Pathology with Clinical and Functional Correlations, Rheumatology, Principles of Internal Medicine (Harrison's), The Pharmacological Basis of Therapeutics (Goodman & Gilman's) and Terkeltaub R. A. Gout: Clinical Practice.

There has been recent renewed interest in hyperuricemia and its effect on cardiovasucular system. The relation between uric acid and cardiovascular disease has been examined in at least 20 epidemiological and clinical studies. Hyperuricemia is associated with cardiovascular impairment over the long term. Recent epidemiological studies have shown that an elevated uric acid is a common feature of the metabolic syndrome, which confers an increased risk for the development of hypertension, ischemic heart disease and stroke. Whether hyperuricemia is a risk factor for cardiovascular disease (causal role), or only a marker part of the metabolic syndrome, is still debated (Watanabe S et al).

The management of gout involves not only treating acute arthritic inflammation and urolithiasis but also lowering urate levels with the goal of preventing recurrent disease and progression. All available systemic therapies for acute gouty arthritis (nonsteroidal anti-inflammatory drugs, systemic corticosteroids and colchine) have significant and potentially severe adverse effects, which may contraindicate their use and justify the need of alternative treatments and preventing occurrence or recurrences by lowering plasma urate level, especially in subjects with serum urate level over 9 mg/dL (530 μmol/L). Reduction of serum uric acid below the saturation level may involve any of several therapeutic strategies. The use of xanthine oxidase inhibitors (e.g. allopurinol) results in decreased production of uric acid, but are also associated with side effects sufficiently severe to often warrant discontinuation of therapy, including e.g. induction of hypersensitivity and adverse drug-drug interactions. The use of uricosuric agents increase the excretion of uric acid thereby reducing the plasma concentration. Among these, probenecid, sulfinpyrazone and benzbromarone are the best known. All are not universally available and have many side effects or contraindications. Activators of peroxisome proliferation—activated receptor as uricosuric agents were proposed in WO 00/47209. Certain insulin sensitivity enhancers of this class, such as troglitazone were proposed to prevent or treat hyperuricemia and related disorders in EP-A-0919232. Little is known about possible relationship between hyperuricemia and cardiovascular diseases and the association of hyperuricemia and such diseases was said to be linked to insulin resistance (Wortmann R L, Gout and hyperuricemia Curr. Opin. Rheumatol 2002 May 14(3):281-6).

Thus, there is a need for further investigation into more potent and safe hypouricemic agents (including uricosuric) to provide new therapeutic treatments offering advantages to existing methods.

Now it was unexpectedly discovered that certain 2,4-pentadienoic acids derivatives, which were disclosed as able to be used in the treatment of dyslipidaemias, artherosclerosis and diabetes, are potent anti-hyperuricemic agents.

These pentadienoic acid derivatives are disclosed in European patent application EP-A-1,140,893 and U.S. Pat. No. 6,596,758 claiming French priority 98 16574 of Dec. 29, 1998 and which are herein incorporated by reference.

The present invention provides a method for the prevention and/or the treatment of hyperuricemia and/or associated disorders or diseases by administering to a subject in need thereof, an effective amount of at least one pentadienoic acid derivative of formula (I).

The diseases associated with hyperuricemia to be treated according to the invention comprise one or several of the following: gout, acute inflammatory arthritis, tophaceous deposition of urate crystals in and around joints, chronic arthritis, deposition of urate crystals in renal parenchyma, urolithiasis, and related renal disease also termed gouty nephropaty.

According to the invention the hyperuricemiae able to be treated do not only comprise primary hyperuricemiae but also secondary hyperuricemiae, such as drug related to hyperuricemiae (e.g. by diuretics, immunosuppressive of cytotoxic agents), or hyperuricemiae related to diverse medical conditions (e.g. nephropaties, myeloproliferative disorders, conditions associated with insulin resistance and transplantations).

The subject to be treated according to the method of the invention may or may not suffer from other diseases or disorders such as for example, dyslipidemias, atherosclerosis or diabetes, or diabetes related disorders.

The invention also provides a method for decreasing serum uric acid levels in a subject by administering to the subject an amount of at least one 2,4-pentadienoic acid derivative of formula (I) effective to reduce the serum uric acid level.

According to a preferred embodiment of the invention, subjects to be treated have serum uric acid levels, before treatment, equal or above 7 mg/dL (420 μmol/L).

Preferably the conditions to be treated are gout or any condition brought about by high levels of uric acid in the joints or kidneys or a serum level over 9 mg/dL (530 μmol/L).

Preferably the amount to be administered to a subject for decreasing the serum level is an amount which achieves normal uric acid levels.

It is also possible to obtain, if needed, serum level reduction up to 80% from the normal serum level in men or women.

The treatment of the invention is preferably conducted by administering the 2,4-pentadienoic acid derivative of formula (I) by the oral route, but it can also be conducted by any other route including parenteral route such as, for example, by injection or infusion.

The treatment according to the invention is preferably performed by administering the effective amount of 2,4-pentadienoic acid derivative according to formula (I) once or twice per day.

The duration of the treatment can easily be adapted to the conditions of the patient, preferably with the aim to obtain a long term normal acid uric serum level.

The invention also provides the use of a pentadienoic acid derivative of formula (I) for the preparation of a medicament for the prevention or treatment of hyperuricemia and/or one or several of the above mentioned associated disorders or diseases, and/or for reducing the serum uric acid level of a subject.

Preferably the use, according to the invention, allows to prepare medicaments for subjects having serum uric acid levels, before treatment, equal or above 7 mg/dL (420 μmol/L), and more preferably, where the conditions to be treated are gout or any condition brought about by high levels of uric acid in the joints or kidneys or a serum level over 9 mg/dL (530 μmol/L).

The use according to the invention is preferably conducted for preparing a medicament suitable for administering the 2,4-pentadienoic acid derivative of formula (I) by the oral route, but it can also be conducted by any other route including parenteral route such as, for example, by injection or infusion.

Preferably the use according to the invention allows to prepare a medicament for administering the effective amount of 2,4-pentadienoic acid or derivative according to formula (I) once or twice per day.

The invention also provides new medical compositions for the treatment of hyperuricemiae and/or the above mentioned associated diseases or disorders which comprise, in a vehicle acceptable for a human, an effective amount of at least one 2,4-pentadienoic acid derivative of formula (I).

Preferably this effective amount is substantially lower than the amount needed for the relevant 2,4-pentadienoic acid derivative used in the treatment of dyslipidemia, atherosclerosis and diabetes. This effective amount is preferably 50% lower and more preferably 90% or even 95% lower.

For example, the effective amount in a dose for a one day administration for an adult human is comprised between 0.15 and 4 mg/kg of a human body, more preferably between 0.3 and 1.mg/kg.

The compounds used according to the invention correspond to the formula (I) below: 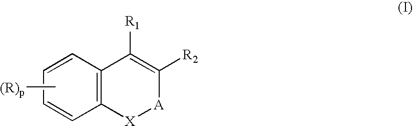
in which:

X represents O or S;

A represents either the divalent radical —(CH₂)ₛ—CO—(CH₂)ₜ— or the divalent radical —(CH₂)ₛ—CR₃R₄—(CH₂)ₜ—

in which radicals s=t=0 or else one of s and t has the value 0 and the other has the value 1;

R₄ represents a hydrogen atom or a (C₁-C₁₅)alkyl group;

R₁ and R₂ independently represent the Z chain defined below; a hydrogen atom; a (C₁-C₁₈)alkyl group; a (C₂-C₁₈)alkenyl group; a (C₂-C₁₈)alkynyl group; a (C₆-C₁₀)aryl group optionally substituted by a halogen atom, by an optionally halogenated (C₁-C₅)alkyl group or by an optionally halogenated (C₃-C₅)alkoxy group; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;

R₃ and R₄ independently takes any one of the meanings given above for R₁ and R₂, with the exception of the Z chain; or else

R₃ and R₄ together form a (C₂-C₆)alkylene chain optionally substituted by a halogen atom or by optionally halogenated (C₁-C₅) alkoxy;

R is chosen from a halogen atom; a cyano group; a nitro group; a carboxy group; an optionally halogenated (C₁-C₁₈)alkoxycarbonyl group; an Rₐ—CO—NH— or RₐR_{b}N—CO— group [in which Rₐ and R_{b} independently represent optionally halogenated (C₁-C₁₈) alkyl; a hydrogen atom; (C₆-C₁₀) aryl or (C₆-C₁₀) aryl(C₁-C₅) alkyl (where the aryl parts are optionally substituted by a halogen atom, by an optionally halogenated (C₁-C₅)alkyl group or by an optionally halogenated (C₁-C₅)alkoxy group); (C₃-C₁₂)cycloalkyl optionally substituted by a halogen atom, by an optionally halogenated (C₁-C₅)alkyl group or by an optionally halogenated (C₁-C₅) alkoxy group]; an optionally halogenated (C₁-C₁₈)alkyl group; optionally halogenated (C₁-C₁₈)alkoxy; and (C₆-C₁₀)aryl, (C₆-C₁₀)aryl(C₁-C₅) alkyl, (C₆-C₁₀)aryloxy, (C₃-C₁₂) cyclo-alkyl, (C₃-C₁₂) cycloalkenyl, (C₃-C₁₂) cycloalkyloxy, (C₃-C₁₂)cycloalkenyloxy or (C₆-C₁₀)aryloxycarbonyl in which the aryl, cycloalkyl and cycloalkenyl parts are optionally substituted by a halogen atom, by optionally halogenated (C₁-C₅)alkyl or by optionally halogenated (C₁-C₅)alkoxy; —OH;

p represents 0, 1, 2, 3 or 4;

Z represents the radical: 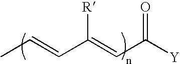
where n is 1 or 2;

The R′ groups independently represent a hydrogen atom; a (C₁-C₅)alkyl group; a (C₆-C₁₀)aryl group optionally substituted by a halogen atom, by an optionally halogenated (C₁-C₅)alkyl group or by optionally halogenated (C₁-C₅)alkoxy; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;

Y represents —OH; (C₁-C₅)alkoxy; or the —NR_{c}R_{d} group (in which R_{c} and R_{d} independently represent a hydrogen atom; (C₃-C₅)alkyl; (C₃-C₈)cycloalkyl optionally substituted by a halogen atom, by optionally halogenated (C₁-C₅)alkyl or by optionally halogenated (C₁-C₅)alkoxy; (C₆-C₁₀)aryl optionally substituted by a halogen atom, by optionally halogenated (C₁-C₅)alkyl or by optionally halogenated (C₁-C₅)alkoxy;

Or Y represents glucomic acid 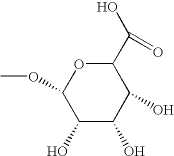
it being understood that one and one alone from R₁ and R₂ represents the Z chain.

The invention is also targeted, depending on the functional groups present in the molecule, at the salts of these compounds with pharmaceutically acceptable acids or bases, and at esters of those compounds.

When the compound of formula (I) comprises an acidic functional group, for example a carboxyl functional group, the latter can form a salt with an inorganic or organic base.

Mention may be made, as example of salts with organic or inorganic bases, of the salts formed with metals and in particular alkali, alkaline earth and transition metals (such as sodium, potassium calcium, magnesium or aluminium) or with bases, such as ammonia or secondary or tertiary amines (such as diethylamine, triethylamine, piperidine, piperazine or morpholine), or with basic amino acids or with osamines (such as meglumine) or with aminoalcohols (such as 3-aminobutanol and 2-aminoethanol).

When the compound of formula (I) comprises a basic functional group, for example a nitrogen atom, the latter can form a salt with an organic or inorganic acid.

The salts with organic or inorganic acids are, for example, the hydrochloride, hydrobromide, sulphate, hydrogensulphate, dihydrogenphosphate, maleate, fumarate, 2-naphthalenesulphonate and para-toluene-sulphonate salts.

The invention also covers the salts which make possible a suitable separation or a suitable crystallization of the compounds of formula (I), such as picric acid, oxalic acid or an optically active acid, for example tartaric acid, dibenzoyltartaric acid, mandelic acid or camphorsulphonic acid.

The formula (I) encompasses all the types of geometric isomers and stereoisomers of the compounds of formula (I).

According to the invention, the term “alkyl” denotes a linear or branched hydrocarbon-comprising radical, such as methyl, ethyl, propyl, isopropyl, butyl, tert-butyl, isobutyl, pentyl, hexyl, heptyl, octyl, nonyl, decyl, undecyl, dodecyl, tridecyl, tetradecyl, pentadecyl, hexadecyl, heptadecyl or octadecyl.

When the alkyl group is substituted by one or more halogen atoms, it is preferable for it to represent perfluoroalkyl and in particular pentafluoroethyl or trifluoromethyl.

The term “alkoxy” denotes an alkyl group as defined above bonded to an oxygen atom. Examples thereof are the methoxy, ethoxy, isopropyloxy, butoxy and hexyloxy radicals.

The term “alkylene group” is understood to mean linear or branched alkylene groups, that is to say bivalent radicals which are linear or branched bivalent alkyl chains.

The term “cycloalkyl” denotes saturated hydrocarbon-comprising groups which can be mono- or polycyclic and comprise from 3 to 12 carbon atoms, preferably from 3 to 8. Preference is more particularly given to monocyclic cycloalkyl groups, such as cyclopropyl, cyclobutyl, cyclopentyl, cyclohexyl, cycloheptyl, cyclooctyl, cyclononyl, cyclodecyl, cyclounaecyl and cyclododecyl.

The term “cycloalkenyl” is understood to mean, according to the invention, a cycloalkyl group exhibiting one or more double bonds.

The term “halogen” is understood to mean a fluorine, chlorine, bromine or iodine atom.

The term “aryl” represents a mono- or bicyclic aromatic hydrocarbon-comprising group comprising 6 to 10 carbon atoms, such as phenyl or naphthyl.

The term “mono- or bicyclic heteroaryl” denotes monocyclic or bicyclic aromatic groups comprising one or more endocyclic heteroatoms. Examples thereof are the furyl, thienyl, pyrrolyl, oxazolyl, isoxazolyl, thiazolyl, isothiazolyl, imidazolyl, pyrazolyl, oxadiazolyl, triazolyl, thiadiazolyl, pyridinyl, pyridazinyl, pyrazinyl, triazinyl, indolizinyl, indolyl, isoindolyl, benzofuryl, benzothienyl, indazolyl, benzimidazolyl, benzothiazolyl, purinyl, quinolyl, quinolizinyl, iqoquinolyl, cinnolinyl, phthalazinyl, quinazolinyl, quinoxalinyl, pteridinyl and benzoxepinyl groups.

Preferred heteroaryls comprise from 4 to 10 carbon atoms and from 1 to 2 heteroatoms.

The alkenyl and alkynyl groups can comprise more than one unsaturation.

The alkenyl groups comprise unsaturations of ethylenic type and the alkynyl groups comprise unsaturations of acetylenic type.

The (C₆-C₁₀) aryl, (C₃-C₈) cycloalkyl, heteroaryl and cycloalkenyl groups are optionally substituted. The expression “optionally substituted by a halogen atom, by an optionally halogenated (C₁-C₅)alkyl group or by an optionally halogenated (C₃-C₅)alkoxy group” indicates that the said aryl, cycloalkyl, heteroaryl and cycloalkenyl groups are optionally substituted by one or more substituents chosen from:
halogen atoms;
alkyl groups optionally substituted by one or more halogen atoms; and
alkoxy groups optionally substituted by one or more halogen atoms.

In the same way, the alkylene chain, when it is substituted, can comprise one or more identical or different substituents chosen from halogen atoms and optionally halogenated alkoxy groups.

The expression “optionally halogenated” means, in the context of the invention, optionally substituted by one or more halogen atoms.

In the context of the present invention, the term “benzoxepine” has been used to denote the benzo[b]oxepine structure of formula:

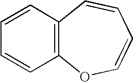

According to the invention, preference is given to the compounds in which A represents the radical:
—(CH₂)ₐ—CR₃R₄—(CH₂)ₜ—
where s, t, R₃ and R₄ are as defined above for the formula (I).

Another preferred group of compounds of formula (I) is composed:

of the compounds in which:
X represents O;
A represents —CR₃R₄— or —CH₂—CR₃R₄— in which the unsubstituted methylene group is bonded to X;
R₁ and R₂ independently represent Z; H; (C₃-C₁₅)alkyl; (C₁-C₁₅)alkenyl; or phenyl optionally substituted by (C₁-C₅)alkyl, (C₁-C₅)alkoxy, a halogen atom or —CF₃;
R₃ takes any one of the meanings given above for R₁ and R₂, with the exception of Z;
R is chosen from (C₁-C₉)alkyl; (C₁-C₅)alkoxy; phenyl or phenylcarbonyl optionally substituted by a halogen atom, (C₁-C₅)alkyl, (C₁-C₅)alkoxy, —CF₃ or —OCF₃; a halogen atom; —CF₃ and —OCF₃;
Z represents the radical: 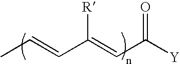
   where n represents 1;
R′ represents (C₁-C₅)alkyl.

Preference is given, among these compounds, to those in which:
X represents O;
A represents —CR₃R₄—;
Z represents

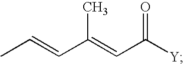

or alternatively those in which:
X represents O;
A represents —CH₂—CR₃R₄— in which the unsubstituted methylene group is bonded to X;
R₁ and R₂ independently represent Z, a hydrogen atom or (C₁-C₅)alkyl; preferably, R₁ represents Z; preferably R₂ represents a hydrogen atom.
R₃ and R₄ independently takes any one of the meanings given above for R₁ and R₂, with the exception of Z;

Preferably R₃ and R₄ independently represent a (C₁-C₅) alkyl group, more preferably a methyl, ethyl, isopropyl, propyl and most preferably a methyl.
Z represents 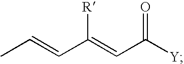
R′ represents (C₃-C₅) alkyl, notably a methyl or phenyl, preferably a methyl.

Preferred meanings of Y are:
—OH
—(C₁-C₅) alkoxy; and
—NR_{c}R_{c} where R_{c} and R_{d} are as defined above for the formula (I).

Very preferably, Y represents —OH or —(C₁-C₅)alkoxy, notably a methoxy, ethoxy, isopropryloxy and most preferably ethoxy.

Preferably R represents a (C₁-C₅) alkoxy, notably a methoxy, ethoxy, isopropyloxy, preferably methoxy.

Likewise, it is preferable for p to have the value 0, 1 or 2. Preferably p represents 1 or 2, most preferably 1.

A particularly preferred group of compounds is composed of the compounds in which:

[X represents O;

A represents —CH₂—CR₃R₄— in which the unsubstituted methylene group is bonded to X

R₁ is Z and R₂ is H;

R₃ and R independently represents a (C₁-C₅) alkyl group;

R₃ is a (C₁-C₅) alkoxy;

Z represents

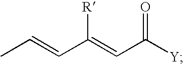

wherein R′ represents a methyl or phenyl; and y represents a (C₁-C₅)alkoxy].

According to a particularly advantageous embodiment of the invention, the compounds of the groups which are preferred defined above are such that p and Y take one of these meanings.

Mention may be made, as example of preferred compounds, of the following compounds:
(2E,4E)-5-(2-pentyl-2H-1-benzopyran-3-yl)-3-methyl-penta-2,4-dienoic acid;
(2Z,4E)-5-(2-pentyl-2H-1-benzopyran-3-yl)-3-methyl-penta-2,4-dienoic acid;
(2E,4E)-5-(2,2-dimethyl-6-methoxy-2H-1-benzopyran-3-yl)-3-methylpenta-2,4-dienoic acid;
(2E,4E)-5-(2H-1-benzopyran-3-yl)-3-methylpenta-2,4-dienoic acid;
(2E,4E)-5-(2,2-dimethyl-2H-1-benzopyran-3-yl)-3-methyl penta-2,4-dienoic acid;
(2Z,4E)-5-(2,2-dimethyl-2H-1-benzopyran-3-yl)-3-methyl-penta-2,4-dienoic acid;
(2E,4E)-5-[2-(non-6-enyl)-2H-1-benzopyran-3-yl]-3-methyl-penta-2,4-dienoic acid;
(2E,4E)-5-(4-phenyl-2H-1-benzopyran-3-yl)-3-methyl-penta-2,4-dienoic acid;
(2E,4E)-5-(6-nonyl-2H-1-benzopyran-3-yl)-3-methyl-penta-2,4-dienoic acid;
(2E,4E)-5-(6-phenyl-2H-1-benzopyran-3-yl)-3-methyl-penta-2,4-dienoic acid;
(2E,4E)-5-(2-nonyl-2H-1-benzopyran-3-yl)-3-methyl-penta-2,4-dienoic acid;
(2E,4E)-5-(4-methyl-2H-1-benzopyran-3-yl)-3-methyl-penta-2,4-dienoic acid;
(2Z,4E)-5-(2H-1-benzopyran-3-yl)-3-methylpenta-2,4-dienoic acid;
(2E,4E)-5-(2-undecanyl-2H-1-benzopyran-3-yl)-3-methyl-penta-2,4-dienoic acid;
(2E,4E)-5-(2-phenyl-2H-1-benzopyran-3-yl)-3-methyl-penta-2,4-dienoic acid;
(2E,4E)-5-(5-methyl-2,3-dihydrobenzoxepin-4-yl)-3-methyl-penta-2,4-dienoic acid;
(2E,4E)-5-(3,3-dimethyl-7-methoxy-2,3-dihydrobenzo-xepin-5-yl)-3-methylpenta-2,4-dienoic acid; and [sic]-(2E,4E)-5-(2,3-dihydrobenzoxepin-4-yl)-3-methylpenta-2,4-dienoic acid;
(2E,4E)-5-(3,3-dimethyl-7-methoxy-2,3-dihydrobenzo-xepin-5-yl)-3-phenylpenta-2,4-dienoic acid;
(2Z,4E)-5-(3,3-dimethyl-7-methoxy-2,3-dihydrobenzo-xepin-5-yl)-3-phenylpenta-2,4-dienoic acid;
(2Z,4E)-5-(3,3-dimethyl-7-methoxy-2,3-dihydrobenzo-xepin-5-yl)-3-methylpenta-2,4-dienoic acid;
(2E,4E)-5-(3,3-dimethyl-7,8-dimethoxy-2,3-dihydrobenzo-xepin-5-yl)-3-methylpenta-2,4-dienoic acid;
(2E,4E)-5-(3,3-dimethyl-2,3-dihydrobenzoxepin-5-yl)-3-methylpenta-2,4-dienoic acid;
(2E,4E)-5-(3,3-dimethyl-2,3-dihydro-7-(para-chloro-benzoyl)benzoxepin-5-yl)-3-methylpenta-2,4-dienoic acid;
(2E,4E)-5-(3,3-dimethyl-7-chloro-2,3-dihydrobenzoxepin-5-yl)-3-methylpenta-2,4-dienoic acid;
(2E,4E)-5-(3,3-dimethyl-7,8-dichloro-2,3-dihydro-benzoxepin-5-yl)-3-methylpenta-2,4-dienoic acid;
(2E,4E)-5-(3,3-dimethyl-7-bromo-2,3-dihydrobenzoxepin-5-yl)-3-methylpenta-2,4-dienoic acid;
(2E,4E)-5-(3,3-dimethyl-1-fluoro-8-chloro-2,3-dihydro-benzoxepin-5-yl)-3-methylpenta-2,4-dienoic acid;
(2E,4E)-5-(3,3-dimethyl-7-fluoro-2,3-dihydrobenzoxepin-5-yl)-3-methylpenta-2,4-dienoic acid;
(2E,4E)-5-(3,3-dimethyl-7-trifluoromethyl-2,3-dihydro-benzoxepin-5-yl)-3-methylpenta-2,4-dienoic acid;
(2E,4E)-5-(3,3-dimethyl-7-phenyl-2,3-dihydrobenzoxepin-5-yl)-3-methylpenta-2,4-dienoic acid;
(2E,4E)-5-(3,3,7-trimethyl-2,3-dihydrobenzoxepin-5-yl)-3-methylpenta-2,4-dienoic acid;
(2E,4E)-5-(3,3-dimethyl-2,3-dihydrobenzoxepin-5-yl)-3-methylpenta-2,4-dienoic acid;
(2E,4E)-5-(9-methoxy-3,3-dimethyl-2,3-dihydrobenzo-xepin-5-yl)-3-methylpenta-2,4-dienoic acid;
   and their pharmaceutically acceptable esters, such as their ethyl esters.

The most preferred compound to be administered in the methods according to the invention, and to be used for the preparation of the medicaments according to the invention, and to be contained as the active principle in the new medicaments is the

(2E,4E)-5-(3,3-dimethyl-7-methoxy-2,3-dihydrobenzoxepin-5-yl)-3-methylpenta-2,4-dienoic acid (compound A), or its pharmaceutically acceptable salts or esters, among which its ethyl ester.

The compounds of formula (I) can be prepared by using one of the processes described in EP-A-1,140,893 or U.S. Pat. No. 6,596,758.

The invention additionally relates to pharmaceutical compositions comprising a pharmaceutically effective amount according to the invention of a compound of formula (I) as defined above in combination with one or more pharmaceutically acceptable vehicles.

These compositions can be administered orally in the form of immediate-release or controlled-release granules, hard gelatin capsules or tablets, intravenously in the form of an injectable solution, transdermally in the form of an adhesive transdermal device, or locally in the form of a solution, cream or gel.

A solid composition for oral administration is prepared by addition of a filler and, if appropriate, a binder, a disintegration agent, a lubricant, a colorant or a flavour enhancer to the active principle and by shaping the mixture as a tablet, a coated tablet, a granule, a powder or a capsule.

Examples of fillers encompass lactose, maize starch, sucrose, glucose, sorbitol, crystalline cellulose and silicon dioxide, and examples of binders encompass poly(vinyl alcohol), poly(vinyl ether), ethylcellulose, methylcellulose, acacia, gum tragacanth, gelatin, shellac, hydroxypropylcellulose, hydroxypropylmethylcellulose, calcium citrate, dextrin and pectin. Examples of lubricants encompass magnesium stearate, talc, polyethylene glycol, silica and hardened vegetable oils. The colorant can be any of those authorized for use in medicaments. Examples of flavour enhancers encompass cocoa powder, mint in herbal form, aromatic powder, mint in oil form, borneol and cinnamon powder. Of course, the tablet or the granule can be suitably coated with sugar, gelatin or the like.

An injectable form comprising the compound of the present invention as active principle is prepared, if appropriate, by mixing the said compound with a pH regulator, a buffer, a suspending agent, a solubilizing agent, a stabilizer, a tonicity agent and/or a preservative and by converting the mixture into a form for intravenous, subcutaneous or intramuscular injection, according to a conventional process. If appropriate, the injectable form obtained can be lyophilized by a conventional process.

Examples of suspending agents encompass methylcellulose, polysorbate 80, hydroxyethyl-cellulose, acacia, gum tragacanth powder, sodium carboxymethylcellulose and polyethoxylated sorbitan monolaurate.

Examples of solubilizing agent encompass castor oil solidified with polyoxyethylene, polysorbate 80, nicotinamide, polyethoxylated sorbitan monolaurate and the ethyl ester of castor oil fatty acid.

In addition, the stabilizer encompasses sodium sulphite, sodium metasulphite and ether, while the preservative encompasses methyl p-hydroxybenzoate, ethyl p-hydroxybenzoate, sorbic acid, phenyl, cresol and chlorocresol.

Examples of compounds useful in the present invention are given in Table 1.

TABLE 1

| | | Characterization |
|---|---|---|
| Example | Chemical formula | physicochemical data |
| 3a | 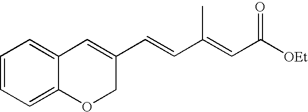 | M.p. = 110-112° C. |
| 3b | 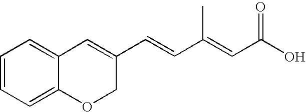 | M.p. = 226-228° C. ¹H NMR (d6-DMSO, 300 MHz) δ (ppm): 2.4 (3H, s), 5.2 (2H, s), 6.0 (1H, s), 6.6 (1H, d, J=16 Hz), 7.1-6.9 (4H, m), 7.3-7.2 (2H, m) |
| 4a | 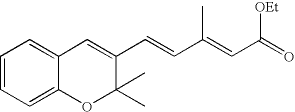 | ¹H NMR (CDCl₃, 300 MHz) δ (ppm): 1.52 (3H, t, J=7.1 Hz), 1.74 (6H, s), 2.56 (3H, d, J=1.1 Hz), 4.41 (2H, q, J=7.1 Hz), 6.09 (1H, s), from 6.66 to 7.36 (7H, m). |
| 4b | 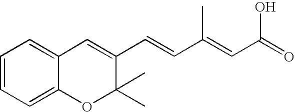 | M.p. = 164-166° C. ¹H NMR (CDCl₃, 300 MHz) δ (ppm): 1.38 (6H, s), 2.5 (3H, s), 6.03 (1H, s), 6.68-7.26 (7H, m). |
| 5a | 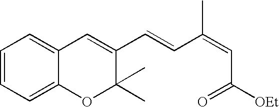 | ¹H NMR (CDCl₃, 300 MHz) δ (ppm): 1.41 (3H, t, J = 7.14 Hz), 1.68 (6H, s), 2.16 (3H, d, J=1.2 Hz), 4.3 (2H, q, J=7.13 Hz), 5.82 (1H, s), 6.58 (1H, d, J=16.35 Hz), from 6.79 to 7.24 (5H, m), 8.3 (1H, d, J=16.2 Hz). |
| 5b | 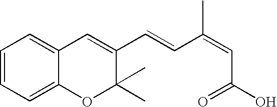 | M.p. = 176° C. ¹H NMR (CDCl₃, 300 MHz) δ (ppm): 1.81 (6H, s) 2.35 (3H, s), 6 (1H, s), 6.77 (1H, d, J=16.2 Hz), 6.93 (1H, s), from 7.02 to 7.4 (4H, m) 8.37 (1H, d, J=16.2 Hz). |
| 6a | 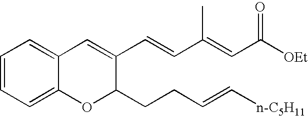 | ¹H NMR (CDCl₃, 300 MHz) δ (ppm): 0.8 (3H, t, J=7 Hz), 1.2-2.3 (15H, m), 2.3 (3H, s), 4.1 (2H, q, J=7 Hz), 5.0 (1H, d, J=14 Hz), (2H, m), 5.8 (1H, s), 6.1 (1H, d, J=16 Hz), 6.4 (1H, s), 6.5 (1H, d, J=16 Hz), (4H, m). |
| 6b | 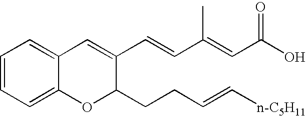 | M.p. = 120-122° C. ¹H NMR (CDCl₃, 300 MHz) δ (ppm): 0.8 (3H, t, J=6.5 Hz), 2.3-0.8 (12H, m), 2.3 (3H, s), 5.1 (1H, d, J=10 Hz), (2H, m), 5.8 (1H, s), 6.2 (1H, d, J=16 Hz), 6.5 (1H, s), 6.6 (1H, d, J=16 Hz), (4H, m). |
| 7a | 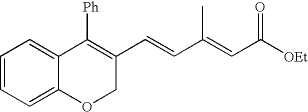 | ¹H NMR (CDCl₃, 300 MHz) δ (ppm): 1.2 (3H, m), 2 (3H, s), from 4.04 (2H, m), 5 (2H, s), 5.7 (1H, s), 6.1 (1H, d, J=16.3 Hz), from 6.47 to 7.39 (10H, m). |
| 7b | 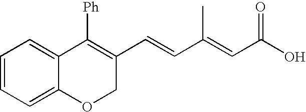 | M.p. = 258-260° C. ¹H NMR (d6-DMSO, 300 MHz) δ (ppm): 1.73 (3H, s), 3.125 (1H, TFA exchangeable), 4.89 (2H, s), 5.67 (1H, s) 6.26 to 6.49 (3H, m), from 6.63 to 6.73 (2H, m), from 6.97 to 7. m), from 7.25 to 7.31 (3H, m). |
| 8a | 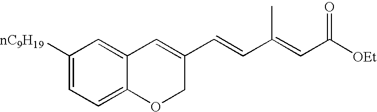 | ¹H NMR (CDCl₃, 300 MHz) δ (ppm): 0.6-1.6 (22H, m), 2.3 (3H, s), 4.1 (2H, q, J=7 Hz), 4.9 (2H, s), 5.8 Hz), 6.5 (1H, s), 6.6 (1H, d, J=16 Hz), 6.7-7.0 (4H, m). |
| 8b | 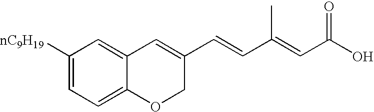 | M.p. = 161-164° C. ¹H NMR (CDCl₃, 300 MHz) δ (ppm): 0.5-1.6 (19H, m), 2.3 (3H, s), 4.9 (2H, s), 5.9 (1H, s), 6.1 (1H, d, J=16 Hz), 6.6 (1H, s), 6.7-6.6 (2H, m), 7.1-6.8 (2H, m). |
| 9a | 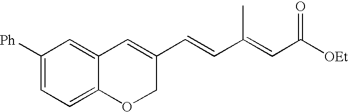 | ¹H NMR (CDCl₃, 300 MHz) δ (ppm): 0.8 (3H, m), 2.3 (3H, s), 4.1 (2H, m), 5.0 (2H, s), 5.8 (1H, s), 6.1 (1H, d, J=16 Hz), 6.5-6.7 (2H, m), 6.8-6.9 (1H, m), 7.1-7.5 (7H, m) |
| 9b | 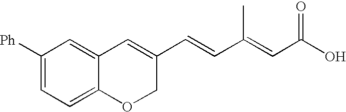 | ¹H NMR (d6-DMSO, 300 MHz) δ (ppm): 2.1 (3H, s), 4.9 (2H, s), 5.8 (1H, s) 6.34 (1H, d, J=16 Hz), 6.8-6.6 (3H, m), 7.5-7.2 (7H, m). |
| 10a | 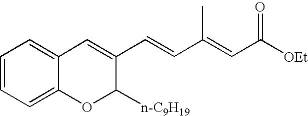 | ¹H NMR (CDCl₃, 300 MHz) δ (ppm): 0.8 (3H, t, J=7 Hz), 1.2-1.7 (19H, m), 2.3 (3H, s), 4.1 (2H, q, J=7 Hz), 5.0 (1H, d, J=10 Hz), 5.8 (1H, s), 6.1 (1H, d, J=16 Hz), 6.4 (1H, s), 6.6 (1H, d, J=16 Hz), 6.8-7.2 (4H, m). |
| 10b | 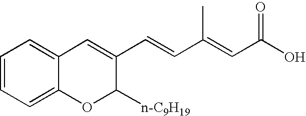 | M.p. = 104-106° C. ¹H NMR (CDCl₃, 300 MHz) δ (ppm): 0.8 (3H, m), (16H, m), 5.0 (1H, d, Hz), 5.8 (1H, s), 6.2 (1H, d, J=16 Hz), 6.5 (1H, s), 6.6 (1H, d, J=16 Hz), 6.9-6.8 (2H, m), 7.1-7.0 (2H, m) |
| 11a | 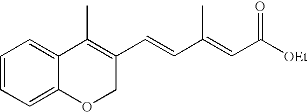 | ¹H NMR (CDCl₃, 300 MHz) δ (ppm): 1.2 (3H, t, J=7 Hz), 2.0 (3H, s), 2.3 (3H, s), 4.1 (2H, q, J=7 Hz), 4.8 (2H, s), 5.8 (1H, s), 6.1 (1H, d, J=16 Hz), (5H, m). |
| 11b | 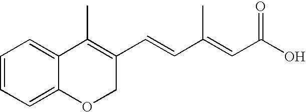 | M.p. = 216-218° C. ¹H NMR (CDCl₃, 300 MHz) δ (ppm): 2.15 (3H, s), 2.3 (3H, s), 4.8 (2H, s), 5.8 (1H, s), 6.2 (1H, d, J=16 Hz), 6.9-6.8 (2H, m), 7.3-7.0 (3H, m). |
| 12a | 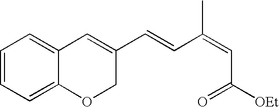 | ¹H NMR (CDCl₃, 300 MHz) δ (ppm): 1.2 (3H, t, J=7 Hz), 2.0 (3H, s), 4.1 (2H, q, J=7 Hz), 5.0 (2H, s), 5.7 (1H, s) (1H, s), 6.5 (1H, s), 6.6 (1H, d, Hz), 6.7-7.2 (4H, m), 7.7 (1H, d, J=16 Hz). |
| 12b | 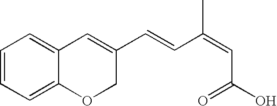 | M.p. = 224-226° C. ¹H NMR (d6-DMSO, 300 MHz) δ (ppm): 2.1 (3H, s), 5.0 (2H, s), 5.8 (1H, s), 7.0-6.8 (4H, m), 7.2-7.18 (2H, m), 7.7 (1H, d, J=16 Hz). |
| 13a | 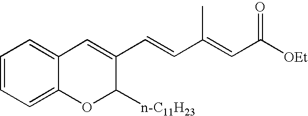 | ¹H NMR (CDCl₃, 300 MHz) δ (ppm): 0.8 (3H, t, J=7 Hz), 1.2-1.8 (23H, m), 2.3 (3H, s), 4.1 (2H, q, J=7 Hz), 5.0 (1H, d, J=10 Hz), 5.8 (1H, s), 6.1 (1H, d, J=16 Hz), 6.4 (1H, s), 6.6 (1H, d, J=16 Hz), 6.8-7.1 (4H, m). |
| 13b | 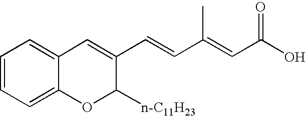 | M.p. = 115-117° C. ¹H NMR (CDCl₃, 300 MHz) δ (ppm): 0.8 (3H, t, J=6.5 Hz), 1.8-1.2 (20H, m), 2.3 (3H, s), 5.0 (1H, d, J=10 Hz), 5.8 (1H, s), 6.2 (1H, d, J=16 Hz), 6.5 (1H, s), 6.6 (1H, d, J=16 Hz), 6.8 (2H, m), 7.0 (1H, d, J=8 Hz), 7.1 (1H, t, J=8 Hz). |
| 14a | 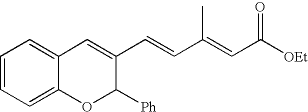 | ¹H NMR (CDCl₃, 300 MHz) δ (ppm): 1.2 (3H, t, J=7 Hz), 2.2 (3H, s), 4.1 (2H, q, J=7 Hz), 5.6 (1H, s), 6.0 (1H, d, J=6 Hz), 6.1 (1H, s) 6.7 (1H, d, J=6 Hz), 6.8 (1H, g),6.8-7 (9H, m) |
| 14b | 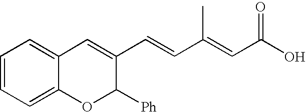 | M.p. = 200-202° C. ¹H NMR (d6-DMSO, 300 MHz) δ (ppm): 2.2 (3H, s), 5.8 (1H, s), 6.36 (1H, s), 6.4 (1H, d, J=16 Hz), 6.8 (1H, d, J=8 Hz), 7.4-6.9 (10H, m) |

Other compound examples are given in the following Table 2.

TABLE 2

| | | Characterization |
|---|---|---|
| Example | Chemical formula | physicochemical data |
| 18 | 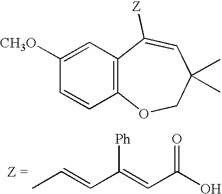 | M.p. 182-184° C. ¹H NMR (CDCl₃, 300 MHz) of the corresponding ethyl ester δ (ppm): 7.4-7.1 (5H, m), 6.85-6.8 (1H, d, J=8.73), 6.7 to 6.45 (3H, m), 6.2 to 6.15 (1H, d, J=15.35 Hz), 5.95 (1H, s), 5.9 (1H, s), 3.95 (2H, q), 3.75 (2H, s), 3.65 (3H, s), 1.1 (6H, s), 1 (3H, t) |
| 19 | 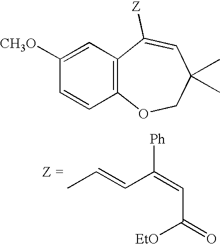 | ¹H NMR (CDCl₃, 300 MHz) δ (ppm): 8 (1H, d, J=15.69 Hz), 7.3 (5H, s), 6.85-6.8 (1H, d, J=8.48 Hz), 6.6 (2H, m), 6.4-6.35 (1H, d, J=15.65 Hz), 6.1 (1H, s), 5.7 (1H, s), 4.15 (2H, q), 3.8 (2H, s), 3.65 (6H, s) |
| 20a | 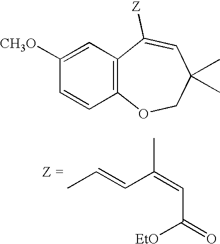 | ¹H NMR (CDCl₃, 300 MHz) δ (ppm): 7.92 (1H, d, J=15.79), 6.7-6.95 (4H, m), 6.08 (1H, s), 5.7 (1H, s), 4.16 (2H, q), 3.84 (2H, s), 3.75 (3H, s), 2.07 (3H, s), 1.28(3H, t), 1.15 (6H, s) |
| 20b | 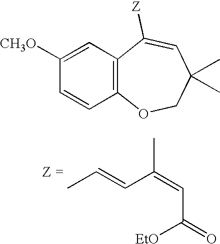 | IR (cm⁻¹) = 2975, 1683, 1493, 1244 ¹H NMR (CDCl₃, 300 MHz) δ (ppm): 7.8-7.9 (lH, d, J=15.66 Hz), 6.9 (1H, d), 6.8-6.6 (3H, m), 6 (1H, s), 5.65 (1H, s), 3.8 (2H, s), 3.7 (3H, s), 2.05 (3H, s), 1.1 (6H, s). |
| 21a | 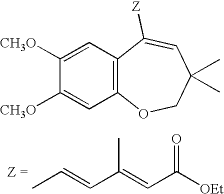 | ¹H NMR (CDCl₃, 300 MHz) δ (ppm): 6.73-6.62 (2H, m), 6.52 (1H, s), 6.40-6.37(1H, d, J=15.4 Hz), 5.80 (1H, s), 5.77 (1H, s), 4.15-4.07 (2H, m), 3.82 (2H, s), 3.75 (3H, s), 3.74 (3H, s), 2.3 (3H, s), 1.25-1.19 (3H, m), 1.08 (6H, s) |
| 21b | 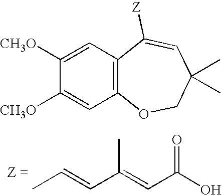 | M.p. = 181-183° C. |
| 22a | 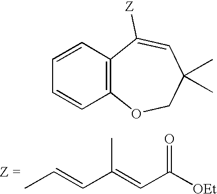 | ¹H NMR (CDCl₃, 300 MHz) δ (ppm): 7.18-6.94 (4H, m), 6.84 (1H, d, J=15.4 Hz), 6.36 (1H, d, J=15.4 Hz), 5.90 (1H, s), 5.77 (1H, s), 4.15-4.07 (2H, m), 3.83 (2H, s), 2.30 (3H, s), 1.24-1.16 (3H, m), 1.09 (6H, s) |
| 22b | 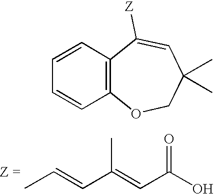 | M.p. = 178-180° C. |
| 23a | 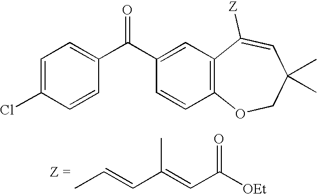 | ¹H NMR (CDCl₃, 300 MHz) δ (ppm): 7.70-7.54 (4H, m), 7.39-7.34 (2H, m), 7.02(1H, d, J=7.9 Hz), 6.67 (1H, d, J=15.4 Hz), 6.36 (1H, d, J=15.4 Hz), 6.09 (1H, s), 5.76 (1H, s), 4.15-4.03 (2H, m), 3.29 (2H, s), 1.96 (3H, s), 1.24-1.20(3H, m), 1.16 (6H, s) |
| 23b | 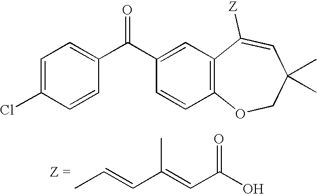 | M.p. = 206-208° C. |
| 24a | 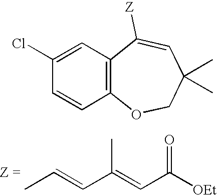 | ¹H NMR (CDCl₃, 300 MHz) δ (ppm): 7.18-7.03 (2H, m), 6.89 (1H, d, J=8.5 Hz), 6.64 (1H, d, J=15.4 Hz), 6.35 (1H, d, J=15.4 Hz), 5.93 (1H, s), 5.78 (1H, s), 4.16-4.08 (2H, m), 3.80 (2H, s), 2.31 (3H, s), 1.25-1.18 (3H, m), 1.08 (6H, s) |
| 24b | 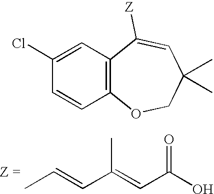 | M.p. = 177-179° C. |
| 25 | 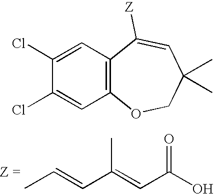 | M.p. = 180° C. ¹H NMR (CDCl₃, 300 MHz) of the corresponding ethyl ester δ (ppm): 7.25 (1H, s), 7 (1H, s), 6.6 (1H, d), 6.3 (1H, d), 5.9 (1H, s), 5.8 (1H, s), 4.15 (2H, m), 3.8 (2H, s), 2.3 (3H, s), 1.2 (3H, t), 1.1 (6H, s). |
| 26a | 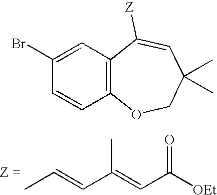 | ¹H NMR (CDCl₃, 300 MHz) δ (ppm): 7.29-6.81 (3H, m), 6.7 (1H, d, J=15.4 Hz), 6.35 (1H, d, J=15.4 Hz), 5.92 (1H, s), 5.79 (1H, s), 4.10 (2H, m), 3.8 (2H, s), 2.31 1.16 (6H, s) |
| 26b | 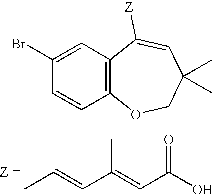 | M.p. = 164-165° C. |
| 27 | 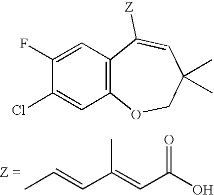 | M.p. = 200° C. ¹H NMR (CDCl₃, 300 MHz) δ (ppm) of the corresponding ethyl ester 7 (2H, m), 6.6 (1H, d, J=d, J=15.45 Hz), 6.3 (1H, d, J=d, J=15.42 Hz) 6 (1H, s), s), s), 5.8 (1H, s), 4.1 (2H, m), 3.8 m), 3.8 (2H, s), 2.3 (3H, s), 1.1 s), 1.1 (6H, s) |
| 28a | 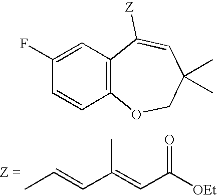 | ¹H NMR (CDCl₃, 300 MHz) δ (ppm): 7.19-6.78 (3H, m), 6.64), 6.64 (1H, d, J=15.4 Hz), 6.34 6.34 (1H, d, J=15.4 Hz), 5.93 5.93 (1H, s), 5.78 (1H, s), 4.15-4.03 (2H, m), 3.80 (2H, s), 2.30 (3H, s), 1.25-1.20 (3H, m), 1.09 (6H, s) |
| 28b | 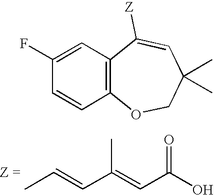 | M.p. = 193-195° C. |
| 29a | 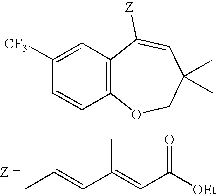 | ¹H NMR (CDCl₃, 300 MHz) δ (ppm): 7.45-7.34 (2H, m), 7.02 (1H, d, J=8.1 Hz), 6.66 (1H, d, J=15.4 Hz), 6.37 (1H, d, J=15.4 Hz), 5.98 (1H, s), 5 s), 4.16-4.09 (2H, m), 3.85 (2H, s), 2.30 (3H, s), 1.25-1.16 (3H, m), 1.11 (6H, s) |
| 29b | 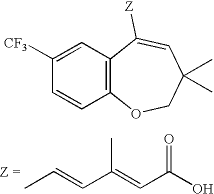 | M.p. = 163-165° C. |
| 30a | 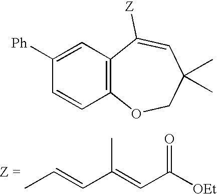 | ¹H NMR (CDCl₃, 300 MHz) δ (ppm): 7-7.6 (8H, m), 6.9 (1H, d, J=15.47 Hz), 6.5 (1H, d, J=15.43 Hz), 6 (1H, s), 5.9 (1H, s), 4 (2H, m), 3.8 (2H, s), 2.24 (3H, s), 1.1 (3H, t), 1.01 (6H, s) |
| 30b | 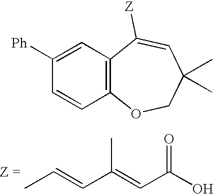 | M.p. = 206-208° C. |
| 31 | 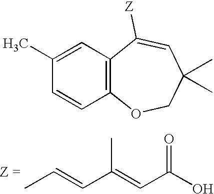 | ¹H NMR (CDCl₃, 300 MHz) δ (ppm): 12.2 (1H, s, exchangeable with CF₃COOD), 7.17-7.06 (2H, m), 6.86-6.97 (2H, m), 6.57 (1H, d, J=15.4 Hz), 6.10 (1H, s), 5.94 (1H, s), 3.89 (2H, s), 2.37 (3H, s), 2.32 (3H, s), 1.17 (6H, s) |
| 32a | 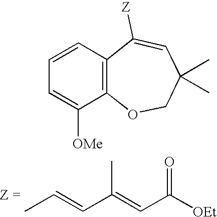 | M.p. = 94° C. ¹H NMR (CDCl₃, 300 MHz) δ (ppm): 6.88-6.68 (4H, m), 6.35 (1H, d, J=15.44 Hz), 5.92 (1H, s), 5.76 (1H, s), 4.10 (2H, m), 3.9 (2H, s), 3.83 (3H, s), 2.3 (3H, s), 1.22 (3H, m), 1.1 (6H, s) |
| 32b | 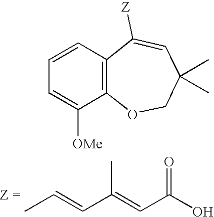 | M.p. = 180-184° C. ¹H NMR (CDCl₃, 300 MHz) δ (ppm): 7.15-6.94 (4H, m), 6.59 (1H, d, J=15.35 Hz), 6.15 (1H, s), 6.0 (1H, s), 4.11 (2H, s), 4.0 (3H, s), 2.51 (3H, s), 1.3 (6H, s) |
| 33 | 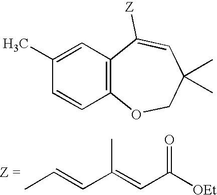 | ¹H NMR (CDCl₃, 300 MHz) δ (ppm): 7.1-6.8 (3H, m), 6.72 (1H, d, J=16 Hz), 6.35 (1H, d, J=15.4 Hz), 5.87 (1H, s), 5.77 (1H, s), 4.15-4.08 (2H, m), 3.80 (2H, s), 2.30 (3H, s), 2.20 (3H, s), 1.25-1.18 (3H, m), 1.08 (6H, s) |
| Ex | Chemical formula | Nomenclature |
| 34A | 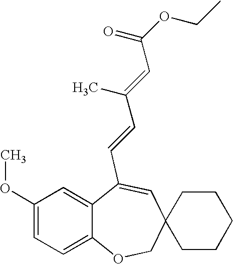 | (2E, 4E)-5-(Spiro[(7-methoxy-2,3- dihydrobenzo [b]-oxepine)-3,1′- cyclohexane]-5-yl)-3-methyl-penta- 2,4-dienoic acid ethyl ester |
| 34B | 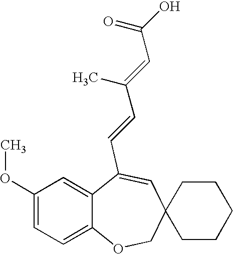 | (2E, 4E)-5-(Spiro[(7-methoxy-2,3- dihydrobenzo [b]-oxepine)-3,1′- cyclohexane]-5-yl)-3-methyl-penta- 2,4-dienoic acid. |
| 35A | 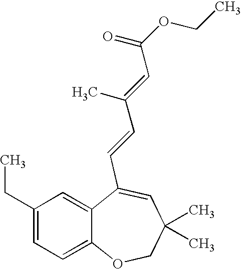 | (2E, 4E)-5-(7-Ethyl-3,3-dimethyl-2,3- dihydrobenzo [b]- oxepine-5-yl)-3 -methylpenta-2,4- dienoic acid ethyl ester |
| 35B | 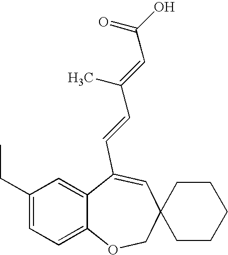 | (2E, 4E)-5-(7-Ethyl-3,3-dimethyl-2,3- dihydrobenzo [b]-oxepin-5-yl)-3- methylpenta-2,4-dienoic acid |
| 36A | 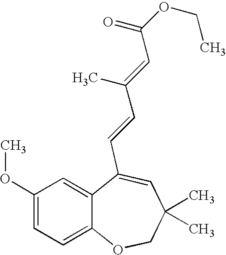 | (2E, 4E)-5-(7-(4-Methoxyphenyl)-3,3- dimethyl-2,3-dihydrobenzo[b]- oxepin-5-yl]-3-methylpenta-2,4- dienoic acid ethyl ester |
| 36B | 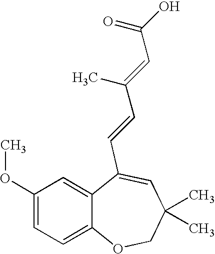 | (2E, 4E)-5-[7-(4-Methoxyphenyl)-3,3- dimethyl-2,3-dihydzobenzo[b]- oxepin-5-yl]-3-methylpenta-2,4- dienoic acid |
| 37A | 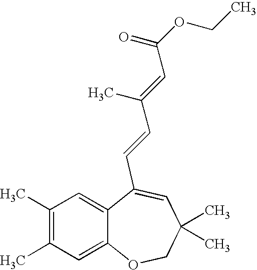 | (2E, 4E)-3-Methyl-5-(3,3,7,8- tetramethyl-2,3-dihydrobenzo[b]- oxepin-5-yl)penta-2,4-dienoic acid ethyl ester |
| 37B | 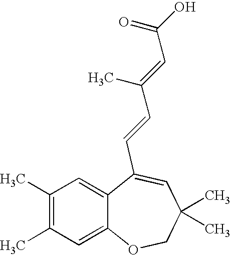 | 3-Methyl-5-(3,3,7,8-tetramethyl-2,3- dihydrobenzo [b]- oxepin-5-yl) penta-2,4-dienoic acid |
| 38A | 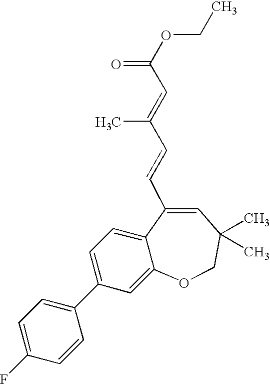 | (2E, 4E)-5-[8-(4-Fluorophenyl)-3,3- dimethyl-2,3-dihydrobenzo[b]- oxepin-5-yl]-3-methylpenta-2,4- dienoic acid ethyl ester |
| 38B | 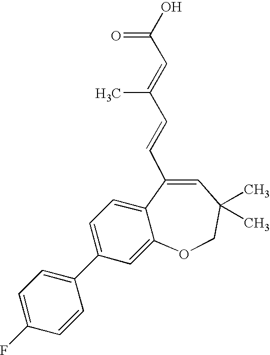 | (2E, 4E)-5-(8-(4-Fluorophenyl)-3,3- dimethyl-2,3-dihydrobenzo[b]- oxepin-5-yl]-3-methylpenta-2,4- dienoic acid |
| 39A | 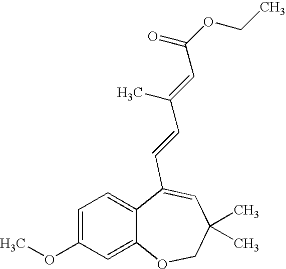 | (2E, 4E)-5-(8-Methoxy-3,3-dimethyl- 2,3-dihydrobenzo[b]- oxepin-5yl)-3-methylpenta-2,4-dienoic acid ethyl ester |
| 39B | 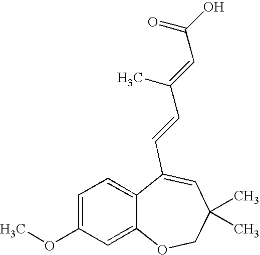 | (2E, 4E)-5-(8-Methoxy-3,3-dimethyl- 2,3-dihydrobenzo[b]- oxepin-5-yl)-3-methylpenta-2,4- dienoic acid |
| 40A | 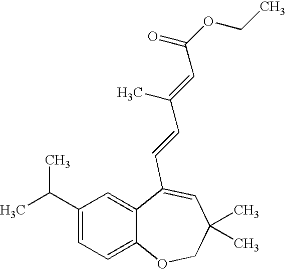 | (2E, 4E)-5-(7-Isopropyl-3,3-dimethyl- 2,3-dihydrobenzo[b]- oxepin-5-yl)-3-methylpenta-2,4- dienoic acid ester. |
| 40B | 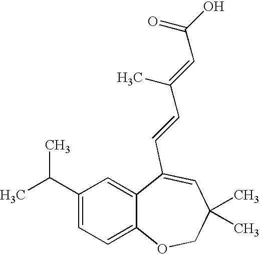 | (2E, 4E)-5-(7-Isopropyl-3,3-dimethyl- 2,3-dihydrobenzo[b]- oxepin-5-yl)-3-methylpenta-2,4- dienoic acid. |
| 41 | 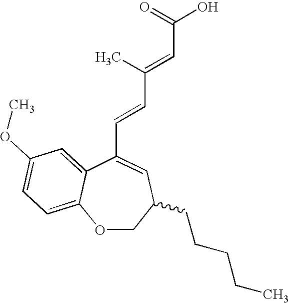 | (2E, 4E)-5-(7-Methoxy-3-pentyl-2,3- dihydrobenzo[b]- oxepin-5-yl)-3-methylpenta-2,4- dienoic acid. |
| 42A | 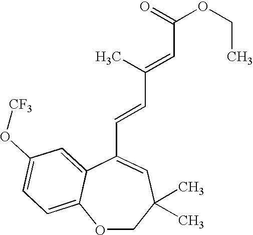 | (2E, 4E)-5-(3,3-Dimethyl-7- trifluoromethoxy-2,3-dihydrobenzz[b]- oxepin-5-yl)-3-methylpenta-2,4- dienoic acid ethyl ester |
| 42B | 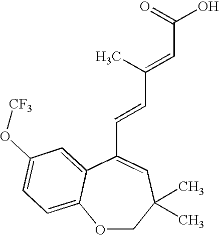 | (2E, 4E)-5-(3,3-Dimethyl-7- trifluoromethoxy-2,3-dihydrobenzo [b]-oxepin-5-yl)-3-methylpenta-2,4- dienoic acid |
| 43A | 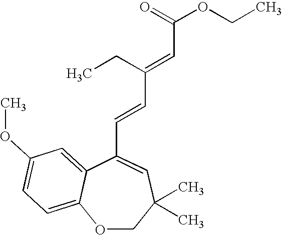 | (2E, 4E)-3-Ethyl-5-(7-methoxy-3,3- dimethyl-2,3-dihydrobenzo[b]-oxepin- 5-yl)penta-2,4-dienoic acid ethyl ester |
| 43B | 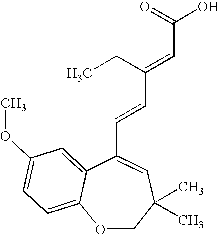 | (2E, 4E)-3-Ethyl-5-(7-methoxy-3,3- dimethyl-2,3-dihydrobenzo[b]- oxepin-5-yl)penta-2,4-dienoic acid |
| 44A | 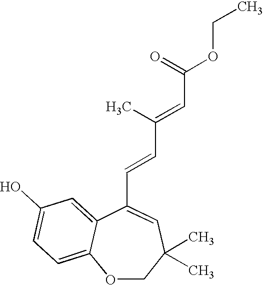 | (2E, 4E)-5-(7-Hydroxy-3,3-dimethyl- 2,3-dihydrobenzo[b]- oxepin-5-yl)-3-methylpenta-2,4- dienoic acid ethyl ester |
| 44B | 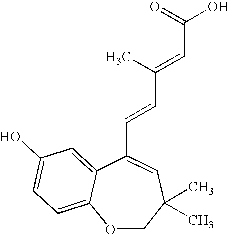 | (2E, 4E)-5-(7-Hydroxy-3,3-dimethyl- 2,3-dihydrobenzo[b]- oxepin-3-yl)-3-methylpenta-2,4- dienoic acid. |
| 45A | 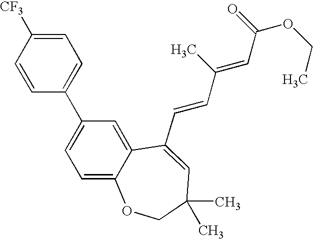 | (2E, 4E)-5-[3,3-Dimethyl-7-(4- (trifluoromethyl)phenyl)-2,3- dihydrobenzo[b]- oxepin-5-yl]-3-methylpenta-2,4- dienoic acid ethyl ester |
| 45B | 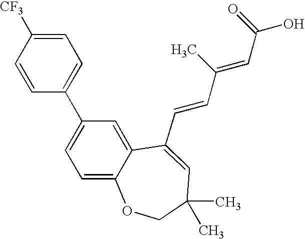 | (2E, 4E)-5-[3,3-Dimethyl-7-(4- (trifluoromethyl)phenyl)-2,3- dihydrobenzo [b]- oxepin-5-yl]-3-methylpenta-2,4- dienoic acid |
| 46A | 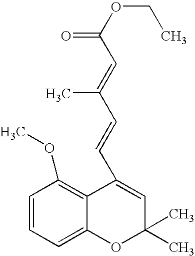 | (2E, 4E)-5-(5-Methoxy-2,2-dimethyl- 2H-chromen-4-yl)-3-methylpenta-2,4- dienoic acid ethyl ester |
| 46B | 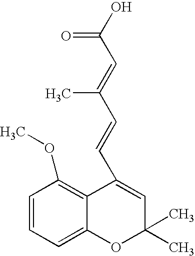 | (2E, 4E)-5-(5-Methoxy-2,2-dimethyl- 2H-chromen-4-yl)-3-methylpenta-2,4- dienoic acid |
| 47A | 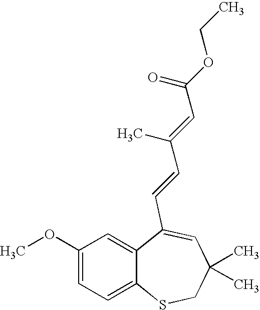 | (2E, 4E)-5-(7-Methoxy-3,3-dimethyl- 2,3-dihydrobenzo[b]- thiepin-5-yl)-3-methylpenta-2,4- dienoic acid ethyl ester |
| 47B | 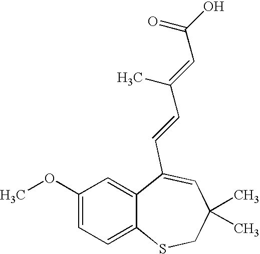 | (2E, 4E)-5-(7-Methoxy-3,3-dimethyl- 2,3-dihydrobenzo[b]- thiepin-5-yl)-3-methylpenta-2,4- dienoic acid |
| 48A | 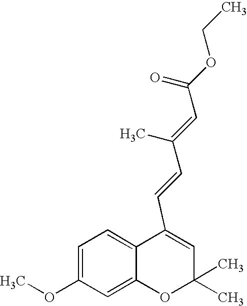 | (2E, 4E)-5-(7-Methoxy-2,2-dimethyl-2H- chromen-4-yl)-3-methylpenta-2,4- dienoic acid ethyl ester |
| 48B | 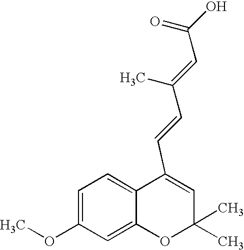 | (2E, 4E)-5-(7-Methoxy-2,2-dimethyl-2H- chromen-4-yl)-3-methylpenta-2,4- dienoic acid |
| 49A | 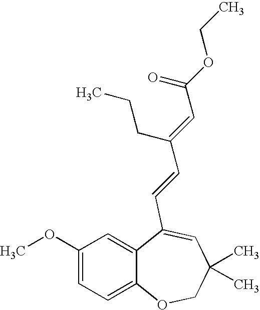 | (2E, 4E)-5-(7-Methoxy-3,3-dimethyl- 2,3-dihydrobenzo[b]- oxepin-5-yJ.)-3-propylpenta-2,4-dienoic acid ethyl ester |
| 49B | 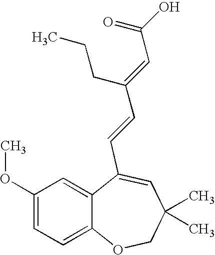 | (2E, 4E)-5-(7-Methoxy-3,3-dimethyl- 2,3-dihydrobenzo[b]- oxepin-5-yl)-3-propylpenta-2,4-dienoic acid |
| 50A | 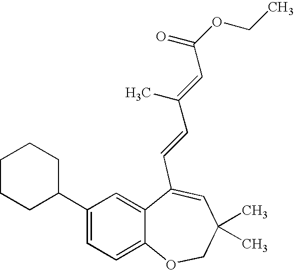 | (2E, 4E)-5-(7-Cyclohexyl-3,3-dimethyl- 2,3-dihydrobenzo[b]- oxepin-5-yl)-3-methylpenta-2,4-dienoic acid ethyl ester |
| 50B | 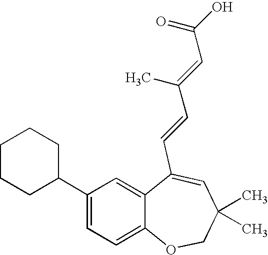 | (2E, 4E)-5-(7-Cyclobexyl-3,3-dimethyl- 2,3-dihydxobenzo[b]- oxepin-5-yl)-3-methylpenta-2,4-dienoic acid |
| 51A | 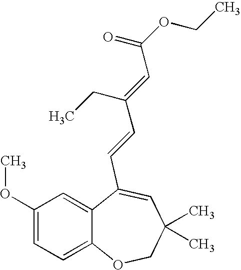 | (2Z, 4E)-3-Ethyl-5-(7-methoxy-3,3- dimethyl-2,3-dihydrobenzo [b]- oxepin-5-yl)penta-2,4-dienoic acid ethyl ester |
| 51B | 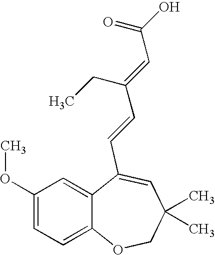 | (2Z, 4E)-3-Ethyl-5-(7-methoxy-3,3- dimethyl-2,3-dihydrobenzo[b]- oxepin-5-yl)penta-2,4-dienoic acid |
| 52A | 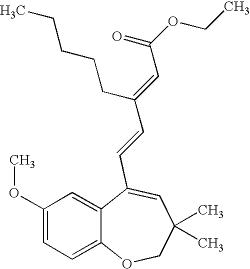 | (2E, 4E)-5-(7-Methoxy-3,3-dimethyl- 2,3-dihydrobenzo[b]- oxepin-5-yl)-3-pentylpenta-2,4- dienoic acid ethyl ester |
| 52B | 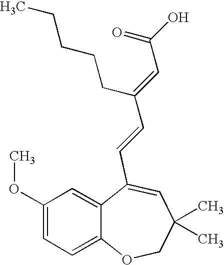 | (2E, 4E)-5-(7-Methoxy-3,3-dimethyl- 2,3-dihydrobenzo[b]- oxepin-5-yl)-3-pentylpenta-2,4- dienoic acid |
| 53A | 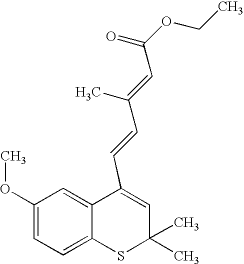 | (2E, 4E)-5-(2,2-Dimethyl- thiochromen-4-yl)-3-methylpenta- 2,4-dienoic acid ethyl ester |
| 53B | 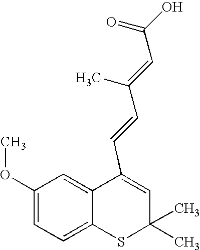 | (2E, 4E)-5-(2,2-Dimethylthio- chromene-4-yl)-3-methylpenta-2,4- dienoic acid |
| 54A | 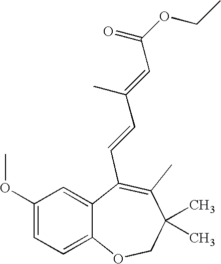 | (2E, 4E)-5-(7-Methoxy-3,3,4- trimethyl-2,3-dihydrobenzo[b]- oxepin-5-yl)-3-methylpenta-2,4- dienoic acid ethyl ester |
| 54B | 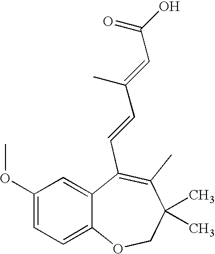 | (2E, 4E)-5-(7-Methoxy-3,3,4- trimethyl-2,3-dihydrobenzo[b]- oxepin-5-yl)-3-methylpenta-2,4- dienoic acid |
| 55A | 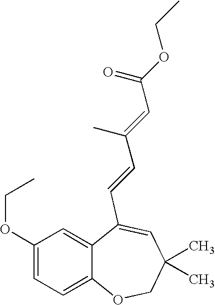 | (2E, 4E)-5-(7-Ethoxy-3,3-dimethyl- 2,3-dihydrobenzo[b]- |
| 55B | 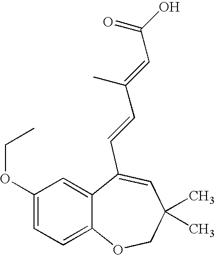 | (2E, 4E)-5-(7-Ethoxy-3,3-dimethyl- 2,3-dihydrobenzo[b]- oxepin-5-yl)-3-methylpenta-2,4- dienoic acid |

The invention will now be described with reference to compound A.

### EXPERIMENTAL EXAMPLE 1

Hypouricemic effect of compound A in healthy male volunteers

Forty-eight healthy male volunteers received orally either EMD or a placebo as a single administration in the morning. Six doses were administered 50 mg, 100 mg, 200 mg, 400 mg, 800 mg and 1200 mg once daily. In each group of dose, 6 subjects received compound A and 2 subjects received a placebo. Plasma uric acid concentrations were assessed in all dose groups.

In all subjects receiving compound A, a dose dependant decrease in plasma uric acid concentration was observed at 24 hours in all groups of dose. The drop of plasma uric acid concentration was around 45% for the lowest tested dose (50 mg) and the maximum effect was observed from the 800 mg dose (80%). No change was observed in placebo.

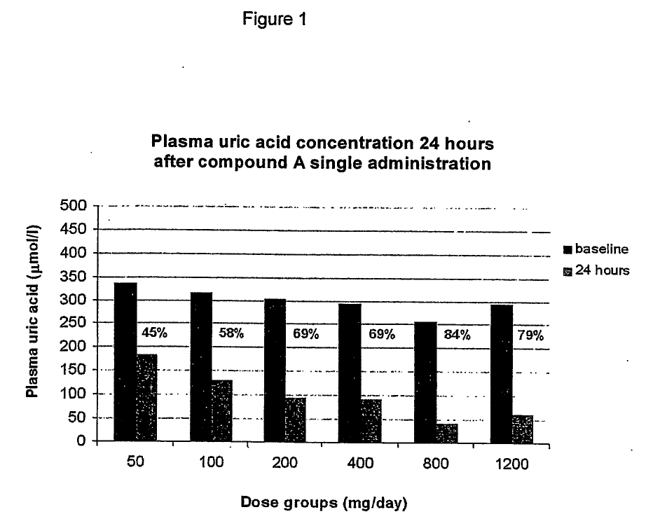

FIG. 1: Mean plasma uric acid concentration in each group of dose before and 24 hours after drug intake (single dose) in subjects receiving compound A.

### EXPERIMENTAL EXAMPLE 2

Sixteen healthy male volunteers received orally either EMD or a placebo as a single administration in the morning (Day 1) followed 3 days later by a repeated administration during 7 days (Day 4 to Day 10); 100 mg and 200 mg were administered once daily. In each group of dose, 6 subjects received compound A and 2 subjects received a placebo. No change was observed in placebo.

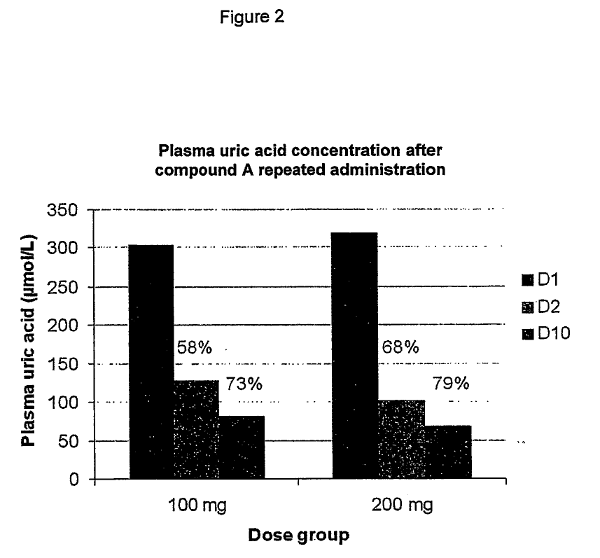

FIG. 2: Mean plasma uric acid concentration per dose group as a function of time and drug dose in subjects receiving repeated administration of compound A.

### REFERENCES

Oxford Textbook of Clinical Nephrology Second Edition. Edited by Davison A. M., Cameron J. S., Grünfeld J. P., Kerr D. N. S., Ritz E. and Winearls C. G. Oxford University Press.
   6. The patient with chronic interstitial disease
   6.4 Uric acid and the kidney. Cameron S. J., Moro F. and Simmonds H. A. 1157-1173
   8. The patient with renal stone disease
   8.1 Aetiological factors in stone formation. Watts R. W. E.,
      Uric acid and urate stones, 1333-1334
   8.2 The medical management of stone disease. Sutton R. A. L.
      Uric acid stones, 1352-1353
   19.2 Handling of drugs in kidney disease. Carmichael D. J. S.
      Hyperuricemia, anti-inflammatory agents, 2671-2672
The Kidney, Brenner & Rector's Fifth Edition. Edited by Brenner B. M. W.B. Saunders Company.
   15. Renal handling of organic anions and cations and renal excretion on uric acid. Sica D. A., Schoolwerth A. C.
      Urate transport 613-617
   28. Acute renal failure. Brady H. R., Brenner B. M., Lieberthal W.
      Acute tubule necrosis. 1204-1207
      Intrinsic renal azotemia, prevention. 1232
   31. Secondary glomerular diseases. Adler S. G., Cohen A. H., Glassock R. J.
      Medications, immunizations, and allergens. 1563-1566
   33. Tubulointerstitial Diseases. Kelly C. J., Neilson E. G.
      Acute interstitial nephritis. 1661-1665
      Chronic interstitial nephritis, uric acid nephropathy. 1669
   34. Toxic Nephropathy. Cronin R., Henrich W. L, Nephrotoxicity of tumor cell lysis 1692
   40. Nephrolithiasis. Asplin J. R., Favus M. J., Coe F. L.
      Hyperuricosuria 1912-1915
      Uric acid stones. 1922-1924
Renal Pathology with Clinical and Functional Correlations, 2^{nd} Edition Edited by Tisher C. C., Brenner B. J. B. Lippincott company
   45. Urate and Uric Acid Nephropathy, Cystinosis, and Oxalosis. Chonko A. M., Richardson W. P.
      Urate and uric acid nephropathy. 1413-1423
Rheumatology, Edited by Klippel J. H., Dieppe P. A., Brooks p, Carette S., Dequeker J., Gerber L. H., Hazleman B. L., Keat A. C. S., Kimberly R. P., Liang M. H., Maini R. N., van de Putte L., Sturrock R. D., Urowitz M. B., Wollheim F. A., Kimberly R. P., Zvaifler N. J. Mosby.
Part 7: Disorders of Bone, Cartilage and Connective Tissue. Dequeker J., van de Putte L.
   12. Crystal arthropathies: Gout. Cohen M. G. & Emmerson B. T. 12.1-12.16
Part 8: Management of Rheumatic Diseases. Brooks P. M. & Gerber L. H
   Pharmacologic approaches, NSAIDs. Brooks P., 10.1-10.6
   Pharmacologic approaches, Systemic corticosteroids in rheumatology. Kirwan J. R. 11.1-11.6
   Pharmacological approaches, Antihyperuricemics. Emmerson B. T. 15.1-15.5
Principles of Internal Medicine, Harrison's 14^{th} Edition edited by Fauci A. S., Martin J. B., Braunwald E., Kasper D. L., Isselbacher K. J., Hauser S. L., Wilson J. D., Longo D. L. McGraw-Hill
Part Ten: Disorders of the Kidney and Urinary Tract
   270: Acute renal failure. Brady H. R., Brenner B. M. 1504-1513
   271: Chronic renal failure. Lazarus J. M., Brenner B. M. 1513-1520
   276: Tubulointerstitial diseases of the kidney. Brenner B. M., Levy E., Hostetter T. H. 1553-1556
Part Thirteen: Endocrinology and Metabolism
   Section 2: Disorders of Intermediary Metabolism 344: Gout and other disorders of purine metabolism. Wortmann R. L. 2158-2166
The Pharmacological Basis of Therapeutics, Goodman & Gilman's, Ninth Edition, Edited by Hardman J. G., Limbird L. E., Molinoff P. B., Ruddon R. W., Goodman Gilman A. McGraw-Hill
Section IV, Autacoïds; Drug Therapy of Inflammation
   27. Analgesic-antipyretic and antiinflammatory agents and drugs employed in the treatment of gout. Insel P. A.
   Drug employed in the treatment of gout 647-650;
   Uricosuric agents 650-653
   Treatment of gout and hyperuricemia 653-655
Terkeltaub R. A. Gout: Clinical Practice. N Engl J. Med 2003; 349:1647-55
Watanabe S, Kang D-H, Feng L, Nakagawa T, Kanellis J, Lan H, Mazzali M, Johnson R. J. Uric Acid, Hominoid Evolution, and the Pathogenesis of Salt-Sensitivity. Hypertension 2002; 40: 355-360

1. The use of a pentadienoic acid derivative of formula (I) for the preparation of a medicament for the prevention or treatment of hyperuricemia and/or one or several associated disorders or diseases, and/or for reducing the serum uric acid level of a subject. 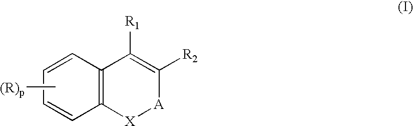
in which:
X represents O or S;
A represents either the divalent radical —(CH₂)ₛ—CO—(CH₂)ₜ— or the divalent radical —(CH₂)ₛ—CR₃R₄—(CH₂)ₜ—
in which radicals s=t=0 or else one of s and t has the value 0 and the other has the value 1;
R₄ represents a hydrogen atom or a (C₁-C₁₅)alkyl group;
R₁ and R₂ independently represent the Z chain defined below; a hydrogen atom; a (C₁-C₁₈)alkyl group; a (C₂-C₁₈)alkenyl group; a (C₂-C₁₈)alkynyl group; a (C₆-C₁₀)aryl group optionally substituted by a halogen atom, by an optionally halogenated (C₁-C₅)alkyl group or by an optionally halogenated (C₁-C₅)alkoxy group; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;
R₃ and R₄ independently takes any one of the meanings given above for R₁ and R₂, with the exception of the Z chain; or else
R₃ and R₄ together form a (C₂-C₆)alkylene chain optionally substituted by a halogen atom or by optionally halogenated (C₁-C₅)alkoxy;
R is chosen from a halogen atom; a cyano group; a nitro group; a carboxy group; an optionally halogenated (C₁-C₁₈)alkoxycarbonyl group; an Rₐ—CO—NH— or RₐR_{b}N—CO— group [in which Rₐ and R_{b} independently represent optionally halogenated (C₁-C₁₈)alkyl; a hydrogen atom; (C₆-C₁₀)aryl or (C₆-C₁₀)aryl(C₁-C₅)alkyl (where the aryl parts are optionally substituted by a halogen atom, by an optionally halogenated (C₁-C₅)alkyl group or by an optionally halogenated (C₁-C₅)alkoxy group); (C₃-C₁₂)cycloalkyl optionally substituted by a halogen atom, by an optionally halogenated C₁-C₅ alkyl [sic] group or by an optionally halogenated (C₁-C₅)alkoxy group]; an optionally halogenated (C₁-C₁₈)alkyl group; optionally halogenated (C₁-C₁₈)alkoxy; and (C₆-C₁₀)aryl, (C₆-C₁₀)aryl(C₁-C₅)alkyl, (C₆-C₁₀)aryloxy, (C₃-C₁₂)cycloalkyl, (C₃-C₁₂)cycloalkenyl, (C₃-C₁₂)cycloalkyloxy or (C₃-C₁₂)cycloalkenyloxy in which the aryl, cycloalkyl and cycloalkenyl parts are optionally substituted by a halogen atom, by optionally halogenated (C₁-C₅)alkyl or by optionally halogenated (C₁-C₅)alkoxy; —OH;
p represents 0, 1, 2, 3 or 4;
Z represents the radical: 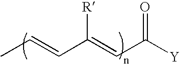
where n is 1 or 2;
the R′ groups independently represent a hydrogen atom; a (C₁-C₅)alkyl group; a (C₆-C₁₀)aryl group optionally substituted by a halogen atom, by an optionally halogenated (C₁-C₅)alkyl group or by optionally halogenated (C₁-C₅)alkoxy; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;
Y represents —OH; (C₁-C₅)alkoxy; or the —NR_{c}R_{d} group (in which R_{c} and R_{d} independently represent a hydrogen atom; (C₁-C₅)alkyl; (C₃-C₈)cycloalkyl optionally substituted by a halogen atom, by optionally halogenated (C₁-C₅)alkyl or by optionally halogenated (C₁-C₅)alkoxy; (C₆-C₁₀)aryl optionally substituted by a halogen atom, by optionally halogenated (C₁-C₅)alkyl or by optionally halogenated (C₁-C₅)alkoxy;
Or Y represents glucomic acid 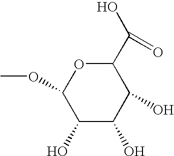
it being understood that one and one alone from R₁ and R₂ represents the Z chain;
and their pharmaceutically acceptable salts with acids or bases, or esters.

2. The use according to claim 1, characterized in that A represents the divalent radical —(CH₂)ₛ—CR₃R₄—(CH₂)ₜ— in which s, t, R₃ and R₄ are as defined in claim 1.

3. The use according to claim 1, characterized in that:
X represents O;
A represents —CR₃R₄— or —CH₂—CR₃R₄— in which the unsubstituted methylene group is bonded to X;
R₁ and R₂ independently represent Z; H; (C₁-C₁₅)alkyl; (C₂-C₁₅)alkenyl; or phenyl optionally substituted by (C₁-C₅)alkyl, (C₁-C₅)alkoxy, a halogen atom or —CF₃;
R₃ and R₄ independently takes any one of the meanings given above for R₁ and R₂, with the exception of Z;
R is chosen from (C₁-C₅)alkyl; (C₁-C₅)alkoxy; phenyl or phenylcarbonyl optionally substituted by a halogen atom, (C₁-C₅)alkyl, (C₁-C₅)alkoxy, —CF₃ or —OCF₃; a halogen atom; —CF₃ and —OCF₃;
Z represents the radical: 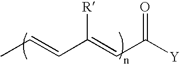
where n represents 1; and
R′ represents (C₁-C₅)alkyl or (C₆-C₁₀)aryl.

4. The use according to claim 1, wherein:
X represents O;
A represents —CH₂—CR₃R₄— in which the unsubstituted methylene group is bonded to X;
R₁ and R₂ independently represent Z, a hydrogen atom or (C₁-C₅)alkyl;
R₃ and R₄ independently takes any one of the meanings given above for R₁ and R₂, with the exception of Z;
Z represents 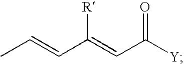
and
R′ represents methyl or phenyl.

5. The use according to claim 1, wherein R1 represents Z.

6. The use according to claim 1, wherein R₂ represents a hydrogen atom.

7. The use according to claim 1, wherein Y is a (C₁-C₅) alkoxy.

8. The use according to claim 1, wherein:
Y represents —OH; (C₁-C₅)alkoxy; or —NR_{c}R_{d} in which R_{c} and R_{d} are as defined in claim 1.

9. The use according to claim 1, wherein R′ is methyl.

10. The use according to claim 1 to 9, wherein R is (C₁-C₅) alkoxy.

11. The use according to claim 1, wherein p represents 0, 1 or 2.

12. The use according to claim 1, wherein:
[X represents O;
A represents —CH₂—CR₃R₄— in which the unsubstituted methylene group is bonded to X;
R₁ is Z and R₂ is H;
R₃ and R₄ independently represents a (C₁-C₅) alkyl group;
R is a (C₁-C₅) alkoxy;
Z represents 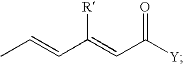
wherein R′ represents a methyl or phenyl; and y represents a (C₁-C₅)alkoxy].

13. The use according to claim 1 wherein said derivative is selected from the group consisting of
(2E,4E)-5-(2-pentyl-2H-1-benzopyran-3-yl)-3-methylpenta-2,4-dienoic acid;
(2Z,4E)-5-(2-pentyl-2H-1-benzopyran-3-yl)-3-methylpenta-2,4-dienoic acid;
(2E,4E)-5-(2,2-dimethyl-6-methoxy-2H-1-benzopyran-3-yl)-3-methylpenta-2,4-dienoic acid;
(2E,4E)-5-(2H-1-benzopyran-3-yl)-3-methylpenta-2,4-dienoic acid;
(2E,4E)-5-(2,2-dimethyl-2H-1-benzopyran-3-yl)-3-methylpenta-2,4-dienoic acid;
(2Z,4E)-5-(2,2-dimethyl-2H-1-benzopyran-3-yl)-3-methylpenta-2,4-dienoic acid;
(2E,4E)-5-[2-(non-6-enyl)₂H-1-benzopyran-3-yl]-3-methylpenta-2,4-dienoic acid;
(2E,4E)-5-(4-phenyl-2H-1-benzopyran-3-yl)-3-methylpenta-2,4-dienoic acid;
(2E,4E)-5-(6-nonyl-2H-1-benzopyran-3-yl)-3-methylpenta-2,4-dienoic acid;
(2E,4E)-5-(6-phenyl-2H-1-benzopyran-3-yl)-3-methylpenta-2,4-dienoic acid;
(2E,4E)-5-(2-nonyl-2H-1-benzopyran-3-yl)-3-methylpenta-2,4-dienoic acid;
(2E,4E)-5-(4-methyl-2H-1-benzopyran-3-yl)-3-methylpenta-2,4-dienoic acid;
(2Z,4E)-5-(2H-1-benzopyran-3-yl)-3-methylpenta-2,4-dienoic acid;
(2E,4E)-5-(2-undecanyl-2H-1-benzopyran-3-yl)-3-methylpenta-2,4-dienoic acid;
(2E,4E)-5-(2-phenyl-2H-1-benzopyran-3-yl)-3-methylpenta-2,4-dienoic acid;
(2E,4E)-5-(5-methyl-2,3-dihydrobenzoxepin-4-yl)-3-methylpenta-2,4-dienoic acid;
(2E,4E)-5-(3,3-dimethyl-7-methoxy-2,3-dihydrobenzoxepin-5-yl)-3-methylpenta-2,4-dienoic acid; and
(2E,4E)-5-(2,3-dihydrobenzoxepin-4-yl)-3-methylpenta-2,4-dienoic acid;
(2E,4E)-5-(3,3-dimethyl-7-methoxy-2,3-dihydrobenzoxepin-5-yl)-3-phenylpenta-2,4-dienoic acid;
(2Z,4E)-5-(3,3-dimethyl-7-methoxy-2,3-dihydrobenzoxepin-5-yl)-3-phenylpenta-2,4-dienoic acid;
(2Z,4E)-5-(3,3-dimethyl-7-methoxy-2,3-dihydrobenzoxepin-5-yl)-3-methylpenta-2,4-dienoic acid;
(2E,4E)-5-(3,3-dimethyl-7,8-dimethoxy-2,3-dihydrobenzoxepin-5-yl)-3-methylpenta-2,4-dienoic acid;
(2E,4E)-5-(3,3-dimethyl-2,3-dihydrobenzoxepin-5-yl)-3-methylpenta-2,4-dienoic acid;
(2E,4E)-5-(3,3-dimethyl-2,3-dihydro-7-(para-chlorobenzoyl)benzoxepin-5-yl)-3-methylpenta-2,4-dienoic acid;
(2E,4E)-5-(3,3-dimethyl-7-chloro-2,3-dihydrobenzoxepin-5-yl)-3-methylpenta-2,4-dienoic acid;
(2E,4E)-5-(3,3-dimethyl-7,8-dichloro-2,3-dihydrobenzoxepin-5-yl)-3-methylpenta-2,4-dienoic acid;
(2E,4E)-5-(3,3-dimethyl-7-bromo-2,3-dihydrobenzoxepin-5-yl)-3-methylpenta-2,4-dienoic acid;
(2E,4E)-5-(3,3-dimethyl-7-fluoro-8-chloro-2,3-dihydrobenzoxepin-5-yl)-3-methylpenta-2,4-dienoic acid;
(2E,4E)-5-(3,3-dimethyl-7-fluoro-2,3-dihydrobenzoxepin-5-yl)-3-methylpenta-2,4-dienoic acid;
(2E,4E)-5-(3,3-dimethyl-7-trifluoromethyl-2,3-dihydrobenzoxepin-5-yl)-3-methylpenta-2,4-dienoic acid;
(2E,4E)-5-(3,3-dimethyl-7-phenyl-2,3-dihydrobenzoxepin-5-yl)-3-methylpenta-2,4-dienoic acid;
(2E,4E)-5-(3,3,7-trimethyl-2,3-dihydrobenzoxepin-5-yl)-3-methylpenta-2,4-dienoic acid;
(2E,4E)-5-(3,3-dimethyl-2,3-dihydrobenzoxepin-5-yl)-3-methylpenta-2,4-dienoic acid;
(2E,4E)-5-(9-methoxy-3,3-dimethyl-2,3-dihydrobenzoxepin-5-yl)-3-methylpenta-2,4-dienoic acid;
and their pharmaceutically acceptable esters.

14. The use according to claim 1 wherein the diseases associated with hyperuricemia to be treated comprise one or several of the following: gout, acute inflammatory arthritis, tophaceous deposition of urate crystals in and around joints, chronic arthritis, deposition of urate crystals in renal parenchyma, urolithiasis, and related renal disease also termed gouty nephropaty.

15. The use according to claim 1 wherein the hyperuricemiae to be treated comprises primary and secondary hyperuricemiae, such as drug related to hyperuricemiae (e.g. by diuretics, immunosuppressive of cytotoxic agents), or hyperuricemiae related to diverse medical conditions (e.g. nephropaties, myeloproliferative disorders, conditions associated with insulin resistance and transplantations).

16. The use according to claim 1 to prepare medicaments for subjects having serum uric acid levels, before treatment, equal or above 7 mg/dL (420% m/L).

17. The use according to claim 16 where the conditions to be treated are gout or any condition brought about by high levels of uric acid in the joints or kidneys or a serum level over 9 mg/dL (530 μmol/L).

18. The use according to claim 1 for preparing a medicament suitable for administering the 2,4-pentadienoic acid derivative of formula (I) by the oral route.

19. The use according to claim 1 for preparing a medicament for administering the effective amount of 2,4-pentadienoic acid or derivative according to formula (I) once or twice per day.

20. The use according to claim 1, wherein the amount of said pentadienoic acid derivative is substantially lower than the amount needed for the relevant derivative as used in the treatment of dyslipidemia, atherosclerosis and diabetes.

21. The use according to claim 20 wherein said amount is at least 50% lower.

22. The use according to claim 21 wherein said amount is at least 90% lower.

23. The use according to claim 1, wherein the amount of said pentadienoic acid derivative is from 0.15 to 4 mg/Kg of human body weight.

24. The use according to claim 23, wherein said amount is from 0.3 to 1.0 mg/Kg human body weight.

25. The use according to claim 1 wherein said derivative is (2E,4E)-5-(3,3-dimethyl-7-methoxy-2,3-dihydrobenzo-xepin-5-yl)-3-methylpenta-2,4-dienoic acid, or its pharmaceutically acceptable salts or esters, among which its ethyl ester.

26. New medical compositions for the treatment of hyperuricemiae and/or the above mentioned associated diseases or disorders and/or for reducing serum uric acid levels which comprise, in a vehicle acceptable for a human, an effective amount of at least one 2,4-pentadienoic acid derivative as defined in claim 1.

27. Medical compositions according to claim 26 wherein this effective amount is substantially lower than the amount needed for the relevant 2,4-pentadienoic acid derivative used in the treatment of dyslipidemia, atherosclerosis and diabetes.

28. Medical compositions according to claim 27 wherein this effective amount is at least 50% lower.

29. Medical compositions according to claim 28 wherein this effective amount is at least 90% lower.

30. Medical compositions according to claim 26 wherein the effective amount in a dose for a one day administration for an adult is from 0.15 to 4 mg/kg of a human body.

31. Medical compositions according to claim 26, wherein said effective amount is from 0.3 to 1.0 mg/Kg of a human body.

32. Medical compositions according to claim 26 formulated for oral administration.

33. A medicament according to claim 26 wherein said derivative is (2E,4E)-5-(3,3-dimethyl-7-methoxy-2,3-dihydrobenzo-xepin-5-yl)-3-methylpenta-2,4-dienoic acid, or its pharmaceutically acceptable salts or esters, among which its ethyl ester.
